(19) United States
(12) Patent Application Publication
(10) Pub. No.: US 2023/0177689 A1
(43) Pub. Date: Jun. 8, 2023

(54) MANAGEMENT DEVICE, EXAMINATION IMAGE EVALUATION METHOD, AND MEDICAL SYSTEM

(71) Applicant: OLYMPUS CORPORATION, Tokyo (JP)

(72) Inventors: Haruhiko SAKAYORI, Tokyo (JP); Kazuyoshi TAMURA, Tokyo (JP)

(73) Assignee: OLYMPUS CORPORATION, Tokyo (JP)

(21) Appl. No.: 18/096,097

(22) Filed: Jan. 12, 2023

Related U.S. Application Data
(63) Continuation of application No. PCT/JP2020/027704, filed on Jul. 16, 2020.

(51) Int. Cl. G06T 7/00; G16H 30/20; G16H 50/20

(52) U.S. Cl. CPC G06T 7/0012; G16H 30/20; G16H 50/20; G06T 2207/10068; G06T 2207/30096

(57) ABSTRACT

An information acquisition unit acquires multiple pieces of image information acquired through image analysis of multiple images captured in an endoscopic examination. An image information storage unit stores the acquired multiple pieces of image information. A number evaluation unit derives the number of captured images of each region based on the stored multiple pieces of image information and assigns, when the derived number of captured images of a region is different from an appropriate number of captured images, a first evaluation value to the evaluation result for the region. An image evaluation unit extracts, from the stored multiple pieces of image information, image information indicating that an image is inappropriate and assigns a second evaluation value to the evaluation result for the region corresponding to the extracted image information.

### CROSS-REFERENCE TO RELATED APPLICATIONS

This application is based upon and claims the benefit of priority from the International Application No. PCT/JP2020/027704, filed on Jul. 16, 2020, the entire contents of which are incorporated herein by reference.

### BACKGROUND

### 1. Field of the Disclosure

The present disclosure relates to a technology for supporting endoscopic examinations.

### 2. Description of the Related Art

In hospitals and other medical facilities, establishing examination protocols for indicating examination procedures is considered to be effective in order to prevent differences in the way of observing organs caused by differences in the experience and skill of doctors (Patent Literature 1, for example). An examination protocol defines regions of an organ to be observed and the order in which the regions are observed. By conducting an examination according to the examination protocol, the doctor can observe an organ without omission and, furthermore, can conduct the examination efficiently, which helps to reduce the burden on the patient.
Patent Literature 1: Japanese Unexamined Patent Application Publication No. 2017-108792

When an inexperienced doctor has conducted an endoscopic examination, it is common that a skilled doctor observes images taken by the inexperienced doctor to double-check whether any finding has been overlooked or whether there is any erroneous diagnosis, for example. At the time, if the number of images taken by the inexperienced doctor is too few, there may be something of which observation is missed, and, if the number of the images is too large, the time required for double-checking becomes long. Also, unclear images or images with abnormal color tone or luminance are not appropriate for observation in the first place and will interfere with the double-checking. Although it would be preferable, by rights, that the skilled doctor who has conducted double-checking also carefully guides the inexperienced doctor based on the check results, skilled doctors are often too busy to spend sufficient time on guidance. Therefore, it is preferable to establish a mechanism for allowing inexperienced doctors to recognize points to be improved or points to be kept in mind based on the results of computerized image analysis of images taken by the inexperienced doctors, without bothering skilled doctors.

### SUMMARY

The present disclosure has been made in view of such a situation, and a purpose thereof is to provide a technology for supporting endoscopic examinations conducted by doctors.

In response to the above issue, a medical system according to one aspect of the present disclosure includes: an information acquisition unit that acquires multiple pieces of image information acquired through image analysis of multiple images captured in an endoscopic examination; an image information storage unit that stores acquired multiple pieces of image information; an appropriate number retaining unit that retains an appropriate number of captured images of each region subject to an endoscopic examination; a first evaluation value retaining unit that retains a first evaluation value, which is an evaluation result of the case where the number of captured images of a region is different from the appropriate number of captured images of the region; a second evaluation value retaining unit that retains a second evaluation value, which is an evaluation result of the case where image information indicates that an image is inappropriate; a number evaluation unit that derives the number of captured images of each region based on the stored multiple pieces of image information and assigns, when the derived number of captured images of a region is different from the appropriate number of captured images, the first evaluation value to the evaluation result for the region; an image evaluation unit that extracts, from the stored multiple pieces of image information, image information indicating that an image is inappropriate and assigns the second evaluation value to the evaluation result for the region included in the extracted image information; and an evaluation result storage unit that stores an evaluation result, which is generated based on the stored multiple pieces of image information and which includes the first evaluation value and the second evaluation value.

Another aspect of the present disclosure also relates to a medical system, which includes: an information acquisition unit that acquires image information through image analysis of a captured image performed by an image analysis device, during an endoscopic examination; an image information storage unit that stores acquired image information; an appropriate number retaining unit that retains an appropriate number of captured images of each region subject to an endoscopic examination; a number evaluation unit that judges, when the number of captured images of a region is smaller than the appropriate number of captured images of the region, that the number of captured images is insufficient; and a notification unit that notifies, when the number evaluation unit has judged that the number of captured images of the region is insufficient, a doctor conducting the endoscopic examination that the number of captured images of the region is insufficient.

Optional combinations of the aforementioned constituting elements, and implementation of the present disclosure in the form of methods, apparatuses, systems, recording media, and computer programs may also be practiced as additional modes of the present disclosure.

### BRIEF DESCRIPTION OF THE DRAWINGS

An embodiment will now be described, by way of example only, with reference to the accompanying drawings which are meant to be exemplary, not limiting, and wherein like elements are numbered alike in several Figures, in which:

FIG. 1 shows a configuration of a medical system according to an embodiment of the present disclosure;

FIG. 2 is a functional block diagram of a management device;

FIG. 3 shows an example of image information;

FIG. 4 shows examples of an appropriate number of captured images;

FIG. 5 shows examples of a first evaluation value;

FIG. 6 shows examples of a second evaluation value;

FIG. 7 shows examples of evaluation results including the first evaluation value and the second evaluation value;

FIG. 8 shows an example of a rank table;

FIGS. 9A and 9B show examples of contents retained in a template retaining unit;

FIG. 10 shows an example of a guidance document; and

FIG. 11 is a functional block diagram of the management device.

### DETAILED DESCRIPTION

The disclosure will now be described by reference to the preferred embodiments. This does not intend to limit the scope of the present disclosure, but to exemplify the disclosure.

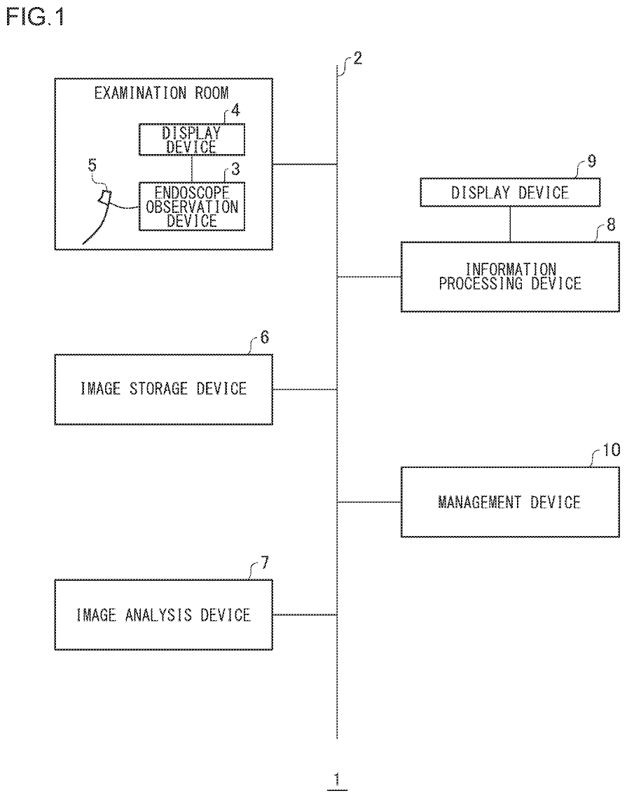

FIG. 1 shows a configuration of a medical system 1 according to an embodiment of the present disclosure. The medical system 1 of the embodiment is installed in a medical facility, such as a hospital, and includes an endoscope observation device 3, an image storage device 6, an image analysis device 7, an information processing device 8, and a management device 10. The endoscope observation device 3, the image storage device 6, the image analysis device 7, the information processing device 8, and the management device 10 are connected by a network 2, such as a local area network (LAN), so as to communicate with each other.

The endoscope observation device 3 is installed in an examination room and connected to an endoscope 5 that is inserted into a patient's digestive tract, so as to display, on a display device 4, images of the inside of the digestive tract captured by the endoscope 5. A doctor observes regions of an organ in sequence according to an examination protocol that defines the examination procedure. The doctor operates a release switch of the endoscope to capture an appropriate number of images defined for each region. For example, the examination protocol specifies that regions of the upper digestive tract should be observed from the esophagus toward the duodenum and that one to two images should be captured in each region. Upon detection of a lesion, the doctor captures an image that includes the lesion. The endoscope observation device 3 captures an image at the timing when the release switch is operated and then transmits the image thus captured to the image storage device 6. Also, the endoscope observation device 3 may transmit multiple captured images collectively to the image storage device 6 after the completion of the examination.

The image storage device 6 is an image server that stores images captured during an endoscopic examination and accumulates images transmitted from the endoscope observation device 3 in relation to an examination order. To a captured image, the date and time of image capturing, an identification number of the endoscope 5, information regarding the examination order, and the like may be added as metadata.

The image analysis device 7 is a device that analyzes images captured during an endoscopic examination and has a computer-aided diagnosis/detection (CAD) function for medical images utilizing artificial intelligence. The image analysis device 7 of the embodiment analyzes captured images and generates image information, as the analysis result, for each captured image. The image information includes organ information, region information, inappropriateness information, and lesion presence/absence information.

(1) Organ Information

The organ information is information that identifies an organ captured in an image. The image analysis device 7 analyzes the captured image to identify which organ is captured in the image. For example, in an upper endoscopy, the organ information indicates one of the “esophagus”, “stomach”, or “duodenum”, which constitute the upper digestive tract.

(2) Region Information

The region information is information that identifies a region captured in an image. The image analysis device 7 analyzes the captured image to identify the organ and also identify the region captured in the image. For example, the “esophagus” is divided into three regions of “upper esophagus,” “middle esophagus,” and “lower esophagus”.

(3) Inappropriateness Information

The inappropriateness information is information indicating that an endoscopic image is not appropriate for observation. For example, for an image that has abnormal color tone or brightness or that is out of focus or blurred, the image analysis device 7 will generate information indicating the reason for inappropriateness (inappropriateness information). Also, since an image with a large amount of residue is not appropriate for observation, upon judging, by image analysis, that there is a large amount of residue, the image analysis device 7 generates the inappropriateness information indicating that the image has a large amount of residue. For images that are clear and appropriate for observation, the inappropriateness information is not generated.

(4) Lesion Presence/Absence Information

When the image analysis device 7 has analyzed an image and judged that a lesion is present, the image analysis device 7 generates, for the image, information indicating that a lesion is included. On the other hand, when the image analysis device 7 has analyzed an image and judged that no lesion is present, the image analysis device 7 generates, for the image, information indicating that no lesion is included. The lesion presence/absence information is used as reference information for image diagnosis by doctors.

When the image analysis device 7 transmits to the image storage device 6 an image provision request that specifies an examination order, the image storage device 6 provides to the image analysis device 7 multiple endoscopic images related to the examination order. For example, upon completion of an examination by an inexperienced doctor, the image analysis device 7 may transmit, to the image storage device 6, an image provision request that specifies the examination order of the examination. Also, when an inexperienced doctor attempts to display the evaluation results of an examination on a display device 9 via the information processing device 8, the image analysis device 7 may transmit, to the image storage device 6, an image provision request that specifies the examination order. Thus, after the completion of an examination, the image analysis device 7 acquires images captured in the examination and analyzes the images. Also, the image analysis device 7 can acquire images captured in an examination during the examination, i.e., in real time, and analyze the images. After the analysis of captured images, the image analysis device 7 transmits image information as the analysis results to the management device 10.

The information processing device 8 may be a personal computer (PC) capable of executing various applications and is used by an inexperienced doctor to create examination reports and the like. After the completion of an examination conducted by an inexperienced doctor, the information processing device 8 of the embodiment displays, on the display device 9, the evaluation results of the examination generated by the management device 10 based on the image information transmitted from the image analysis device 7. By viewing the evaluation results, the inexperienced doctor can recognize the points to be improved.

The management device 10 of the embodiment has multiple functions to support endoscopic examinations conducted by inexperienced doctors. One of the functions of the management device 10 is to generate, after the completion of an examination conducted by an inexperienced doctor, the evaluation results including evaluation of captured images, using the image information analyzed by the image analysis device 7. Another function of the management device 10 is to notify, before the start of an examination conducted by an inexperienced doctor, the inexperienced doctor of points to be kept in mind during the examination to be conducted, based on the evaluation results of examinations conducted by the inexperienced doctor in the past. Yet another function of the management device 10 is to perform, during an examination, notification of messages regarding appropriate image capturing. In the following, each function of the management device 10 will be described.

### EXAMPLE 1

In Example 1, the management device 10 has the function to generate evaluation results including evaluation of captured images.

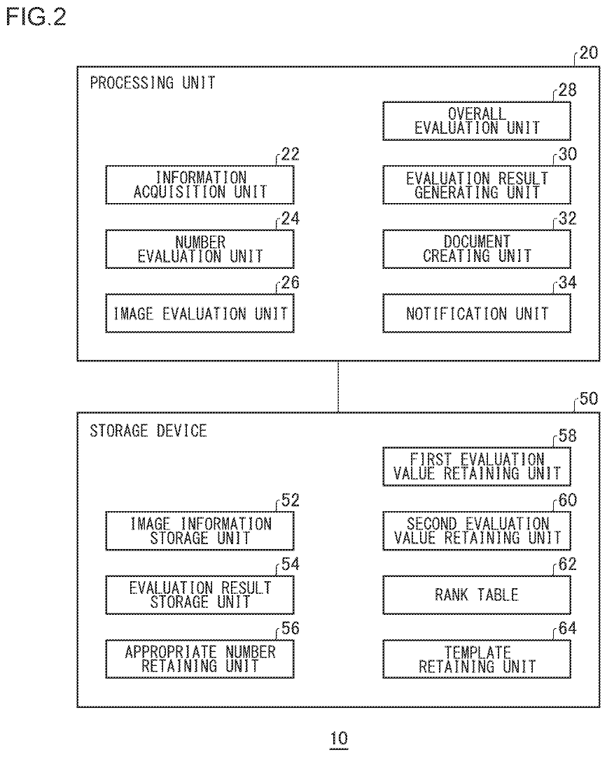

FIG. 2 shows functional blocks of the management device 10 in Example 1. The management device 10 includes a processing unit 20 and a storage device 50. The processing unit 20 includes an information acquisition unit 22, a number evaluation unit 24, an image evaluation unit 26, an overall evaluation unit 28, an evaluation result generating unit 30, a document creating unit 32, and a notification unit 34. The storage device 50 includes an image information storage unit 52, an evaluation result storage unit 54, an appropriate number retaining unit 56, a first evaluation value retaining unit 58, a second evaluation value retaining unit 60, a rank table 62, and a template retaining unit 64.

The configuration shown in FIG. 2 may be implemented by an arbitrary processor, memory, auxiliary storage device, and other LSIs in terms of hardware, and by a memory-loaded evaluation generating application program or the like in terms of software. FIG. 2 illustrates functional blocks implemented by coordination thereof. Therefore, it will be understood by those skilled in the art that these functional blocks may be implemented in a variety of forms by hardware only, software only, or a combination thereof.

At the time of completion of an examination conducted by an inexperienced doctor, the image storage device 6 stores all images captured during the examination. As described above, the endoscope observation device 3 may transmit to the image storage device 6 an image captured by a doctor operating the release switch, each time the capturing is performed, or the endoscope observation device 3 may accumulate the captured images and transmit the captured images collectively to the image storage device 6 upon the completion of the examination.

Upon notification of the completion of an examination from the endoscope observation device 3, the image analysis device 7 transmits to the image storage device 6 the image provision request that specifies the examination order of the examination. The image storage device 6 transmits all captured images related to the examination order to the image analysis device 7. The image analysis device 7 analyzes all the captured images using the medical image CAD function and generates the image information for each captured image. The image analysis device 7 transmits, to the management device 10, multiple pieces of image information related to the examination order.

In the management device 10, the information acquisition unit 22 acquires, from the image analysis device 7, multiple pieces of image information acquired through the image analysis of multiple captured images. The image information storage unit 52 stores the multiple pieces of image information thus acquired by the information acquisition unit 22, in relation to the examination order.

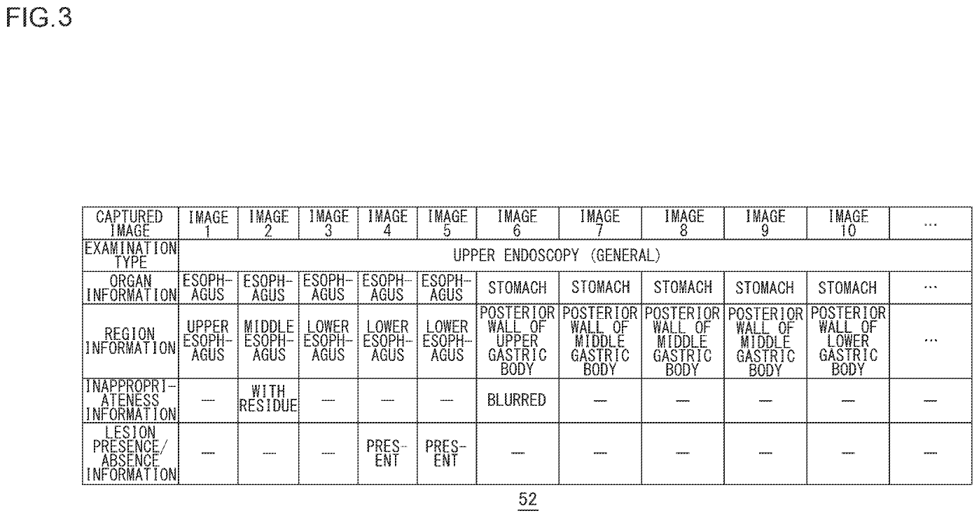

FIG. 3 shows an example of image information stored in the image information storage unit 52. The image information is configured to include the organ information, region information, inappropriateness information, and lesion presence/absence information regarding a captured image. Note that FIG. 3 shows part of the image information of images captured during an upper endoscopy (general) and that, in actuality, the image information of captured images of the duodenum is also included. The image information is treated as metadata of the captured image.

For example, for IMAGE 1, the organ information of “ESOPHAGUS”, the region information of “UPPER ESOPHAGUS”, the inappropriateness information of “-”, and the lesion presence/absence information of “-” is set as the image information. For IMAGE 2, the organ information of “ESOPHAGUS”, the region information of “MIDDLE ESOPHAGUS”, the inappropriateness information of “WITH RESIDUE”, and the lesion presence/absence information of “-” is set as the image information.

The “INAPPROPRIATENESS INFORMATION” is information that indicates the reason why the image is not appropriate for observation, and “WITH RESIDUE” set for IMAGE 2 means that the image is not suitable for observation because a large amount of residue can be seen in the image. Also, “BLURRED” set for IMAGE 6 means that the image is not suitable for observation because the endoscope camera was moved at the capturing and the resulting image is blurred. If no inappropriateness information is set for all images captured during an examination, it means that all the images captured during the examination are clear and fine for observation.

The “LESION PRESENCE/ABSENCE INFORMATION” is information that indicates whether or not the image includes a lesion and that is used as reference information for image diagnosis by doctors. In the example shown in FIG. 3, information indicating the presence of a lesion is set for IMAGE 4 and IMAGE 5. With the lesion presence/absence information thus set, doctors can observe IMAGE 4 and IMAGE 5 more carefully during image diagnosis.

The appropriate number retaining unit 56 retains an appropriate number of captured images of each region subject to endoscopic examinations.

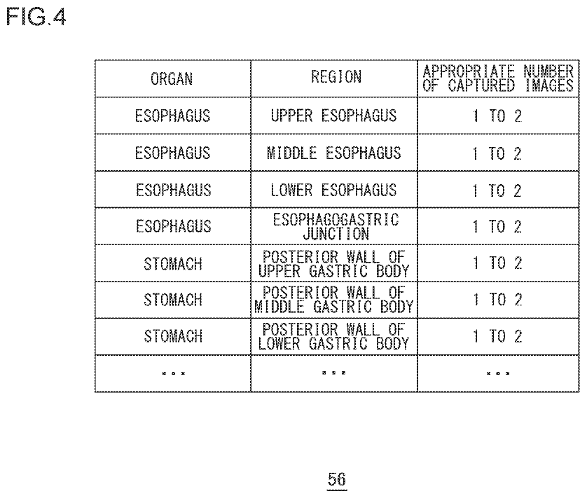

FIG. 4 shows examples of the appropriate number of captured images stored in the appropriate number retaining unit 56. The appropriate number retaining unit 56 retains an appropriate number of images to be captured (appropriate number of captured images) in relation to each region. In this example, the appropriate number retaining unit 56 retains “1 to 2” as the appropriate number of captured images for each of the upper esophagus, middle esophagus, lower esophagus, esophagogastric junction, posterior wall of the upper gastric body, posterior wall of the middle gastric body, and posterior wall of the lower gastric body.

The examination protocol that the doctors comply with defines at least the order of regions to be observed and the appropriate number of captured images of each region. In Example 1, the examination protocol specifies that regions of the upper digestive tract should be observed from the esophagus toward the duodenum and also specifies a range of an appropriate number of images to be captured at each region. Therefore, the doctors have to capture the appropriate number of images at each region, and, if the number of captured images of a region is smaller than the appropriate number of captured images or, conversely, larger than the appropriate number of captured images, the image capturing of the region will be judged to be inappropriate, and the evaluation for the image capturing of the region will be lowered.

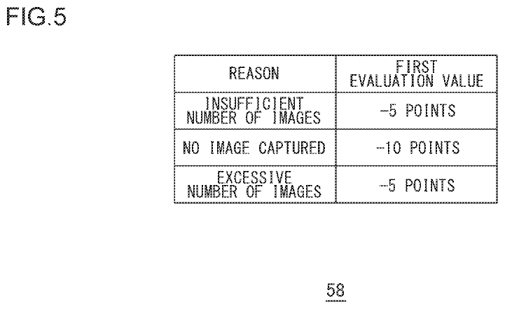

FIG. 5 shows examples of a first evaluation value stored in the first evaluation value retaining unit 58. The first evaluation value retaining unit 58 retains the first evaluation value, which is an evaluation result of the case where the actual number of captured images of a region is different from the appropriate number of captured images of the region. In the example shown in FIG. 5, the first evaluation value of −5 points is set as the evaluation result for the image capturing of a region when the actual number of captured images is smaller or larger than the appropriate number of captured images of the region. Also, the first evaluation value of −10 points is set as the evaluation result for the image capturing of a region of which an image has not been captured (or has been forgotten to be captured).

The number evaluation unit 24 compares the actual number of captured images of each region with the appropriate number of captured images of the region retained in the appropriate number retaining unit 56, and, when the actual number of captured images differs from the appropriate number of captured images, the number evaluation unit 24 assigns the first evaluation value to the evaluation result for the region. Since the appropriate number of captured images shown in FIG. 4 is 1 to 2, the case where the actual number of captured images is smaller than the appropriate number of captured images corresponds to a situation where the actual number of captured images is 0. However, when the actual number of captured images is 0, the number evaluation unit 24 assigns the first evaluation value for the case of not performing image capturing (−10 points) instead of the first evaluation value for the case of an insufficient number of images (−5 points). Therefore, when the minimum appropriate number of captured images is set to 2 or larger and when the actual number of captured images is 1 or larger and smaller than the minimum appropriate number of captured images, the number evaluation unit 24 judges that the actual number of captured images is smaller than the appropriate number of captured images and assigns the first evaluation value for the case of an insufficient number of images (−5 points).

In specific, the number evaluation unit 24 derives the actual number of captured images of each region based on multiple pieces of image information stored in the image information storage unit 52. With reference to the image information shown in FIG. 3, the actual number of captured images of each region is derived as follows:

| | |
|---|---|
| Upper esophagus | 1 image |
| Middle esophagus | 1 image |
| Lower esophagus | 3 images |
| Esophagogastric junction | 0 images |
| Posterior wall of upper gastric body | 1 image |
| Posterior wall of middle gastric body | 3 images |
| Posterior wall of lower gastric body | 1 image |

When the derived actual number of captured images of a region is different from the appropriate number of captured images, based on the reason for the difference, the number evaluation unit 24 assigns the first evaluation value to the evaluation result for the region. More specifically, when the actual number of captured images is insufficient or excessive, −5 points are assigned to the evaluation result for the image capturing of the region. When the actual number of captured images is 0, i.e., when image capturing has not been performed, −10 points are assigned to the evaluation result for the image capturing of the region.

With reference to FIG. 4, since the appropriate number of captured images of each region is 1 to 2, the regions of which the actual number of captured images is different from the appropriate number of captured images are the “lower esophagus”, “esophagogastric junction”, and “posterior wall of the middle gastric body”. With regard to the “lower esophagus” and “posterior wall of the middle gastric body”, the number of captured images is excessive, and, with regard to the “esophagogastric junction”, image capturing has not been performed. Therefore, the number evaluation unit 24 may assign−5 points to each of the evaluation results for the “lower esophagus” and the “posterior wall of the middle gastric body” and may assign −10 points to the evaluation result for the “esophagogastric junction”.

Meanwhile, even when the actual number of captured images of a region is larger than the appropriate number of captured images, if a lesion is present in the region, the number evaluation unit 24 may not assign the first evaluation value to the evaluation result for the region. This is because, since a region where a lesion exists needs to be observed in more detail, it is preferable that a larger number of images are captured. In Example 1, since the presence of a lesion in the “lower esophagus” is confirmed, it is preferable that the number evaluation unit 24 provides the evaluation result of no point deduction, instead of the evaluation result of −5 points, for the “lower esophagus”.

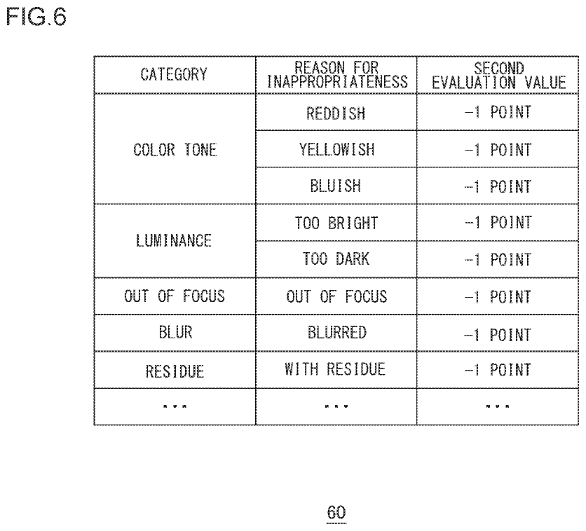

FIG. 6 shows examples of a second evaluation value stored in the second evaluation value retaining unit 60. The second evaluation value retaining unit 60 retains the second evaluation value, which is an evaluation result of the case where the image information indicates that the image is inappropriate. In the example shown in FIG. 6, the second evaluation value of −1 point is set for each reason why the image is inappropriate for observation. Although all the second evaluation values are set to the same point to be deduced in this example, the second evaluation values may also be set to different points to be deduced.

The image evaluation unit 26 extracts image information indicating that the image is inappropriate, from the multiple pieces of image information stored in the image information storage unit 52. With reference to the image information shown in FIG. 3, the image information of each of IMAGE 2 and IMAGE 6 includes the inappropriateness information. Accordingly, the image evaluation unit 26 assigns the second evaluation value to the evaluation result for the region included in the extracted image information of each of IMAGE 2 and IMAGE 6. More specifically, the image evaluation unit 26 assigns −1 point to each of the evaluation results for the “middle esophagus” and the “posterior wall of the upper gastric body”.

Based on the multiple pieces of image information stored in the image information storage unit 52, the evaluation result generating unit 30 generates the evaluation results. The evaluation result generating unit 30 includes, in each evaluation result, the first evaluation value assigned by the number evaluation unit 24 and the second evaluation value assigned by the image evaluation unit 26. The evaluation result storage unit 54 stores the evaluation results thus generated by the evaluation result generating unit 30.

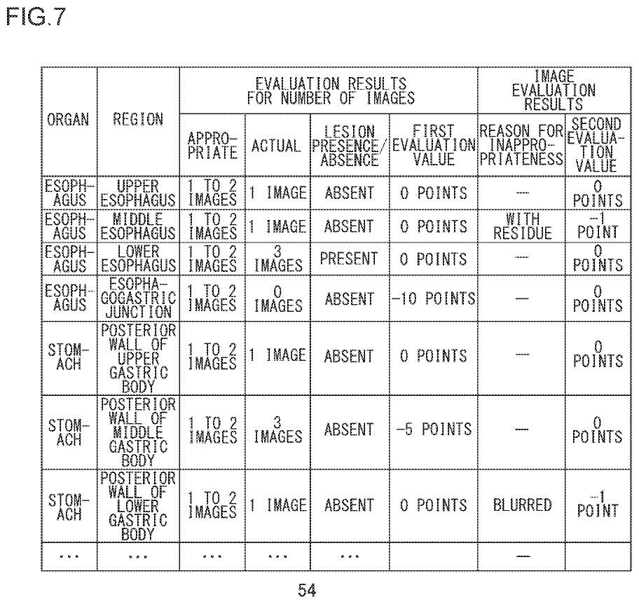

FIG. 7 shows examples of the evaluation results including the first evaluation value and the second evaluation value. The evaluation result generating unit 30 generates evaluation results obtained through evaluation of images of each region captured by a doctor. In the “EVALUATION RESULTS FOR NUMBER OF IMAGES” column, the appropriate number of captured images, the actual number of captured images, the presence or absence of a lesion, and the first evaluation value are displayed. As described above, with regard to the “lower esophagus”, although the actual number of captured images exceeds the appropriate number of captured images, the evaluation of no point deduction is displayed because a lesion is present in the region. In the “IMAGE EVALUATION RESULTS” column, the reason for inappropriateness of the image and the second evaluation value are displayed.

The evaluation result storage unit 54 stores the evaluation results, which are generated based on multiple pieces of image information and which each include the first evaluation value and the second evaluation value. After the completion of an examination, an inexperienced doctor can access the management device 10 via the information processing device 8 to obtain the evaluation results and can also display the evaluation results on the display device 9.

The overall evaluation unit 28 sums the first evaluation value and the second evaluation value included in an evaluation result. In Example 1, the evaluation of the captured images is performed using a point deduction method, and hence more total minus points indicate lower evaluation. Based on the sum total of the first evaluation value and the second evaluation value, the overall evaluation unit 28 determines whether the conducted endoscopic examination was good or bad.

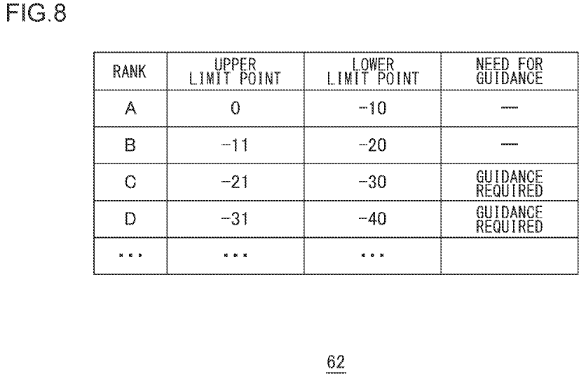

FIG. 8 shows an example of the rank table 62. The rank table 62 is a table in which ranks are related to the sum totals. Ranks A and B indicate being acceptable, and a rank C or lower indicates being rejected. The overall evaluation unit 28 identifies a rank from a sum total, and, when it is the rank A or B, the overall evaluation unit 28 judges that the endoscopic examination was conducted favorably. When the identified rank is C or lower, on the other hand, the overall evaluation unit 28 judges that the endoscopic examination was not conducted favorably. The rank table 62 shown in FIG. 8 specifies that, when the rank is C or lower, guidance by a skilled doctor should be performed, which means, for example, that a supervising doctor is required to provide guidance to the inexperienced doctor. Such operation may be determined in a medical facility as appropriate, and, for example, an inexperienced doctor who has been rated at the rank C multiple times may be required to receive guidance from a supervising doctor.

The document creating unit 32 has a function to create a guidance document for an inexperienced doctor, based on an evaluation result stored in the evaluation result storage unit 54. By setting the document creating unit 32 to automatically create the guidance document based on an evaluation result, points to be improved can be precisely conveyed to the inexperienced doctor, without bothering a supervising doctor.

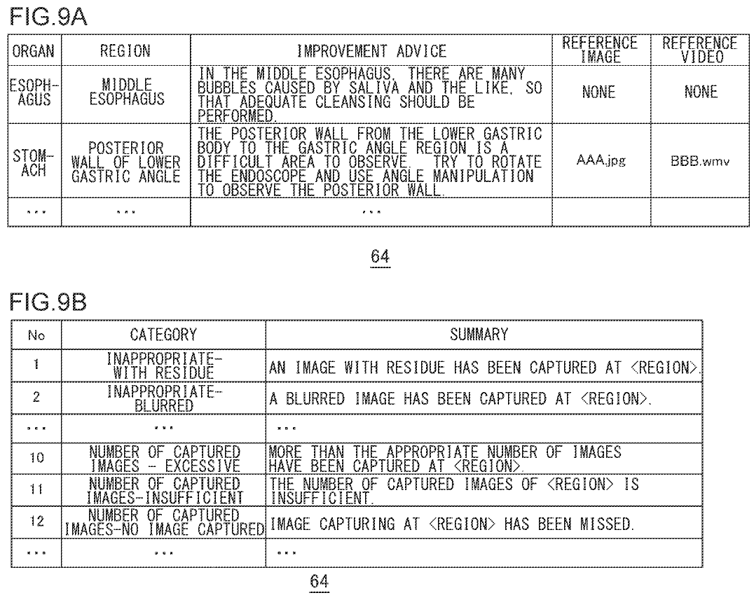

FIG. 9A shows an example of a sentence template retained in the template retaining unit 64. The sentence template is prepared for each region, and the document creating unit 32 inserts, into a guidance document, improvement advice associated with a region for which inappropriateness information is set. A reference image and/or a reference video for supporting the improvement advice may also be registered.

FIG. 9B shows an example of a conversion table retained in the template retaining unit 64. The conversion table is prepared for each reason for inappropriateness regarding a captured image or the number of images, and the document creating unit 32 generates a summary regarding a region for which the first evaluation value and the second evaluation value are set and inserts the summary into a guidance document.

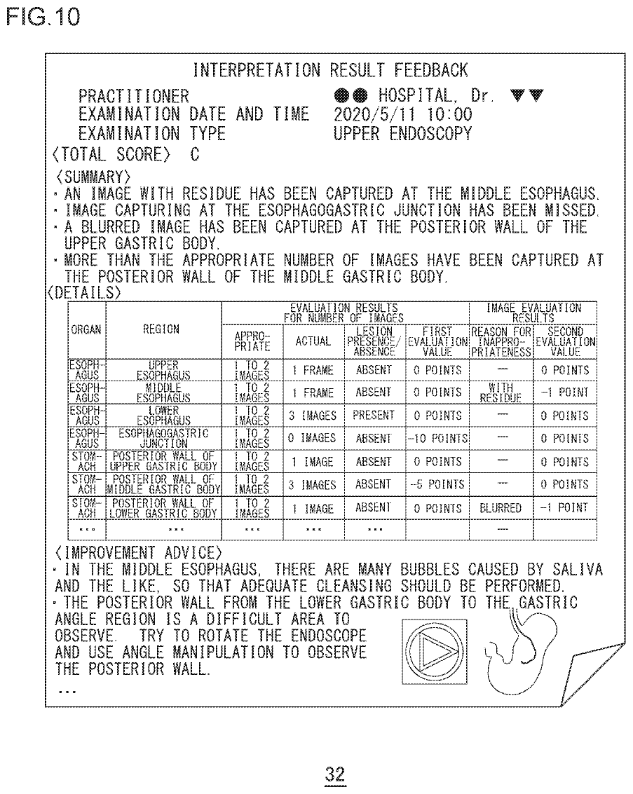

FIG. 10 shows an example of the guidance document created by the document creating unit 32. The document creating unit 32 creates the guidance document that includes the rank rated by the overall evaluation unit 28, the summary and the improvement advice created by the document creating unit 32, and the evaluation results generated by the evaluation result generating unit 30. After the completion of an examination, an inexperienced doctor can access the management device 10 via the information processing device 8 to obtain the guidance document created by the document creating unit 32 and can also display the guidance document on the display device 9. Since the guidance document includes not only the evaluation results but also the improvement advice and the like, the inexperienced doctor can make use of the guidance document for future examinations.

As described above, in Example 1, the management device 10 generates an evaluation result regarding image capturing after the completion of an examination. This evaluation result is used by the inexperienced doctor to review the examination and may also be used for advice when the inexperienced doctor conducts a new examination.

When a doctor starts an endoscopic examination, the notification unit 34 performs notification of advice based on an evaluation result included in the evaluation results stored in the evaluation result storage unit 54. The advice may be displayed on the display device 4 connected to the endoscope observation device 3 in the examination room or may be displayed on a display device of a terminal device (not illustrated) installed in the examination room.

When a doctor starts an endoscopic examination in the examination room, the notification unit 34 acquires from the evaluation result storage unit 54 the evaluation results regarding the past endoscopic examinations conducted by the doctor and generates advice regarding regions for which the first evaluation value and the second evaluation value have been assigned. For example, the notification unit 34 may create an advice sentence, such as “In the previous examination, image capturing of the esophagogastric junction was missed, so don't forget to capture the image.” or “In the previous examination, a blurred image was captured at the posterior wall of the upper gastric body, so please capture the image with caution.”, and may display the advice sentence on a display device in the examination room. The notification unit 34 may output the advice sentence as sound. By recognizing failures in the past examinations before the start of an examination, the doctor will be more careful in conducting the examination.

### EXAMPLE 2

In Example 2, the management device 10 has the function to evaluate an image captured during an endoscopic examination in real time.

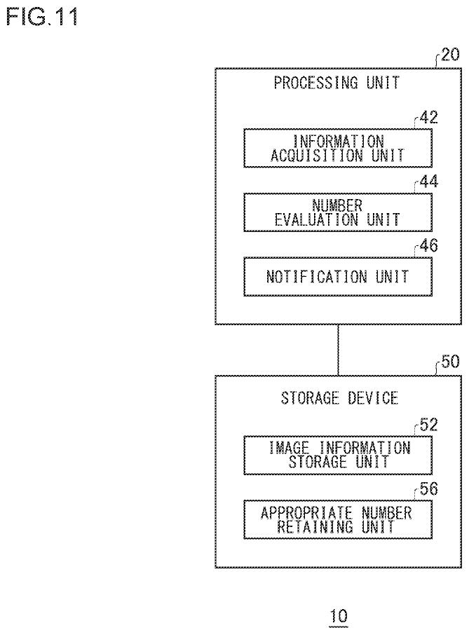

FIG. 11 shows functional blocks of the management device 10 in Example 2. The management device 10 includes the processing unit 20 and the storage device 50. The processing unit 20 includes an information acquisition unit 42, a number evaluation unit 44, and a notification unit 46. The storage device 50 includes the image information storage unit 52 and the appropriate number retaining unit 56.

The configuration shown in FIG. 11 may be implemented by an arbitrary processor, memory, auxiliary storage device, and other LSIs in terms of hardware, and by a memory-loaded evaluation generating application program or the like in terms of software. FIG. 11 illustrates functional blocks implemented by coordination thereof. Therefore, it will be understood by those skilled in the art that these functional blocks may be implemented in a variety of forms by hardware only, software only, or a combination thereof.

During an endoscopic examination conducted by an inexperienced doctor, the endoscope observation device 3 transmits, to the image storage device 6 and the image analysis device 7, an image captured by a doctor operating the release switch, each time the capturing is performed. The image analysis device 7 analyzes a captured image using the medical image CAD function to generate the image information for the captured image and transmits, to the management device 10, the image information in relation to the corresponding examination order. Thus, in Example 2, the image information is generated immediately after an image is captured during an examination and transmitted to the management device 10.

In the management device 10, when the information acquisition unit 42 acquires the image information from the image analysis device 7, the image information storage unit 52 stores the image information thus acquired. As in Example 1, the appropriate number retaining unit 56 in Example 2 also retains the appropriate number of captured images of each region subject to endoscopic examinations (see FIG. 4). The number evaluation unit 44 counts the number of captured images of each region to judge whether or not the number is smaller than the appropriate number of captured images of the region. The examination protocol that the doctors comply with in Example 2 defines the order of regions to be observed and the appropriate number of captured images of each region. More specifically, the examination protocol specifies that regions of the upper digestive tract should be observed from the esophagus toward the duodenum and also specifies a range of an appropriate number of images to be captured at each region.

Based on the examination protocol, the number evaluation unit 44 monitors whether the appropriate number of images are captured at each region. For example, it is assumed here that the examination protocol specifies that the images should be captured in the order of the upper esophagus, middle esophagus, lower esophagus, esophagogastric junction, posterior wall of the upper gastric body, posterior wall of the middle gastric body, posterior wall of the lower gastric body, and so on. After the start of an endoscopic examination, if the information acquisition unit 42 has acquired the image information of the upper esophagus, the image information of the middle esophagus, and the image information of the esophagogastric junction in this order, the number evaluation unit 44 will judge that the number of captured images of the lower esophagus is smaller than the appropriate number of captured images (one to two) of the lower esophagus (in this case, image capturing has not been performed) and will judge that the number of captured images is insufficient.

Thus, the number evaluation unit 44 has the function to monitor the number of captured images of each region according to the examination protocol and judge, when the number of captured images of a region is smaller than the appropriate number of captured images of the region, that the number of captured images of the region is insufficient. The judgment result is transmitted immediately to the notification unit 46, which then notifies the doctor conducting the endoscopic examination that the number of captured images of the region is insufficient. The notification unit 46 may display the fact of the number of captured images being insufficient on the display device 4 connected to the endoscope observation device 3 in the examination room or on a display device of a terminal device (not illustrated) installed in the examination room.

In endoscopic submucosal dissection (ESD), a lesion is cut out using special instruments, and, during the procedure, the order of image capturing of regions may be changed. Therefore, only when a specific examination, such as the upper routine examination, is conducted, the number evaluation unit 44 may monitor the number of captured images of each region.

When the image information includes inappropriateness information indicating that the image is inappropriate, the notification unit 46 notifies the doctor conducting the endoscopic examination that the captured image is inappropriate. At the time, it is preferable to provide notification in line with the reason for the inappropriateness. For example, if a blurred image has been captured at the posterior wall of the upper gastric body, the notification may be given such as “A blurred image was captured at the posterior wall of the upper gastric body. Please capture the image again with caution.”

The present disclosure has been described with reference to an embodiment. The embodiment is intended to be illustrative only, and it will be obvious to those skilled in the art that various modifications to a combination of constituting elements or processes could be developed and that such modifications also fall within the scope of the present disclosure. In the embodiment, the image storage device 6 and the image analysis device 7 installed in a medical facility are described. However, the functions of the image storage device 6 and/or the image analysis device 7 may be implemented by a cloud server connected to the Internet.

What is claimed is:

1. A management device comprising a processor including hardware, wherein the processor is configured to:
acquire a plurality of pieces of image information acquired through image analysis of a plurality of images captured in an endoscopic examination;
derive, based on the acquired plurality of pieces of the image information, the number of captured images of each region subject to the endoscopic examination;
assign, when the derived number of captured images of the region is different from an appropriate number of captured images, a first evaluation value to an evaluation result for the region;
extract, from the acquired plurality of pieces of the image information, image information indicating that an image is inappropriate;
assign a second evaluation value to the evaluation result for the region corresponding to the extracted image information; and
generate the evaluation result that includes the first evaluation value and the second evaluation value, based on the acquired plurality of pieces of the image information.

2. The management device according to claim 1, wherein the processor is configured to sum the first evaluation value and the second evaluation value included in the evaluation result.

3. The management device according to claim 2, wherein the processor is configured to determine whether a conducted endoscopic examination was good or bad, based on the sum total of the first evaluation value and the second evaluation value.

4. The management device according to claim 1, wherein, even when the number of captured images of the region is larger than the appropriate number of captured images, if a lesion is present in the region, the processor is configured not to assign the first evaluation value to the evaluation result for the region.

5. The management device according to claim 1, wherein the processor is configured to create a guidance document for a doctor based on the evaluation result.

6. The management device according to claim 1, wherein, when a doctor starts an endoscopic examination, the processor is configured to perform notification of advice based on the evaluation result.

7. The management device according to claim 1, wherein, when the number of captured images of a region is smaller than the appropriate number of captured images of the region, the processor is configured to notify a doctor conducting the endoscopic examination that the number of captured images of the region is insufficient.

8. The management device according to claim 1, wherein, when the image information includes information indicating that an image is inappropriate, the processor is configured to perform notification of the captured image being inappropriate.

9. An examination image evaluation method for evaluating an examination image, the examination image evaluation method comprising:
acquiring a plurality of pieces of image information acquired through image analysis of a plurality of images captured in an endoscopic examination;
deriving, based on the acquired plurality of pieces of the image information, the number of captured images of each region subject to the endoscopic examination;
assigning, when the derived number of captured images of the region is different from an appropriate number of captured images, a first evaluation value to an evaluation result for the region;
extracting, from the acquired plurality of pieces of the image information, image information indicating that an image is inappropriate;
assigning a second evaluation value to the evaluation result for the region corresponding to the extracted image information; and
generating the evaluation result that includes the first evaluation value and the second evaluation value, based on the acquired plurality of pieces of the image information.

10. The examination image evaluation method according to claim 9, comprising:
summing the first evaluation value and the second evaluation value included in the evaluation result; and
determining whether a conducted endoscopic examination was good or bad, based on the sum total.

11. The examination image evaluation method according to claim 9, comprising:
not assigning the first evaluation value to the evaluation result for the region, even when the number of captured images of the region is larger than the appropriate number of captured images, if a lesion is present in the region.

12. The examination image evaluation method according to claim 9, comprising:
creating a guidance document for a doctor, based on the evaluation result.

13. The examination image evaluation method according to claim 9, comprising:
performing notification of advice based on the evaluation result when a doctor starts an endoscopic examination.

14. A medical system comprising a storage device and a processor including hardware, the storage device retains an appropriate number of captured images of each region subject to an endoscopic examination; a first evaluation value, which is an evaluation result of the case where the number of captured images of a region is different from the appropriate number of captured images of the region; and a second evaluation value, which is an evaluation result of the case where image information indicates that an image is inappropriate;
the processor is configured to:
acquire a plurality of pieces of image information acquired through image analysis of a plurality of images captured in an endoscopic examination;
derive, based on the acquired plurality of pieces of the image information, the number of captured images of each region subject to the endoscopic examination;
assign, when the derived number of captured images of the region is different from an appropriate number of captured images, a first evaluation value to an evaluation result for the region;
extract, from the acquired plurality of pieces of the image information, image information indicating that an image is inappropriate;
assign a second evaluation value to the evaluation result for the region corresponding to the extracted image information; and
generate the evaluation result that includes the first evaluation value and the second evaluation value, based on the acquired plurality of pieces of the image information.

15. The medical system according to claim 14, wherein the processor is configured to sum the first evaluation value and the second evaluation value included in the evaluation result.

16. The medical system according to claim 15, wherein the processor is configured to determine whether a conducted endoscopic examination was good or bad, based on the sum total of the first evaluation value and the second evaluation value.

17. The medical system according to claim 14, wherein, even when the number of captured images of the region is larger than the appropriate number of captured images, if a lesion is present in the region, the processor is configured not to assign the first evaluation value to the evaluation result for the region.

18. The medical system according to claim 14, wherein the processor is configured to create a guidance document for a doctor based on the evaluation result.

19. The medical system according to claim 14, wherein, when a doctor starts an endoscopic examination, the processor is configured to perform notification of advice based on the evaluation result.

20. The medical system according to claim 14, wherein, when the number of captured images of a region is smaller than the appropriate number of captured images of the region, the processor is configured to notify a doctor conducting the endoscopic examination that the number of captured images of the region is insufficient.
